(19) United States
(12) Patent Application Publication
(10) Pub. No.: US 2013/0108559 A1
(43) Pub. Date: May 2, 2013

(54) Hydrogen Peroxide Toothpaste With Improved Stability

(71) Applicant: THE PROCTER & GAMBLE COMPANY, Cincinnati, OH (US)

(72) Inventors: Sanjeev Midha, Mason, OH (US); Michael Jude Leblanc, Cincinnati, OH (US); George Endel Deckner, Cincinnati, OH (US)

(73) Assignee: THE PROCTER & GAMBLE COMPANY, Cincinnati, OH (US)

(21) Appl. No.: 13/666,181

(22) Filed: Nov. 1, 2012

Related U.S. Application Data
(60) Provisional application No. 61/554,062, filed on Nov. 1, 2011.

(51) Int. Cl. A61K 8/92; A61Q 11/00

(52) U.S. Cl. 424/52

(57) ABSTRACT

Aqueous oral care compositions containing hydrogen peroxide and having improved stability, such compositions containing at least 35%, by weight of the composition, of water and further including a peroxide compatible abrasive such as fused silica, calcium pyrophosphate and/or dicalcium phosphate, a gel network, and a protection system containing sodium acid pyrophosphate, disodium phosphate, and/or phosphoric acid.

### FIELD OF THE INVENTION

The present invention relates to an oral composition containing sodium fluoride or MFP, hydrogen peroxide, abrasive, and a gel network.

### BACKGROUND OF THE INVENTION

An effective oral composition can maintain and preserve tooth appearance by removing dental stains and polishing the teeth. It may clean and remove external debris as well, which can aid the prevention of tooth decay and promote gingival health.

Abrasives in oral compositions aid in the removal of the tightly adherent pellicle film to which dental stains affix. Pellicle film usually comprises a thin acellular, glycoprotein-mucoprotein coating, which adheres to the enamel within minutes after teeth are cleaned. The presence of various food pigments lodged within the film accounts for most instances of teeth discoloration. An abrasive may remove the pellicle film with minimal abrasive damage to oral tissue, such as the dentin and enamel.

In addition to cleaning, it may be desirable for abrasive systems to provide polishing of tooth surfaces, as polished surfaces may be more resistant to ectopic deposition of undesirable components. Tooth appearance may be improved by imparting a polished character to the teeth, because the surface roughness, that is, its polish, affects light reflectance and scattering, which integrally relate to the teeth's visual appearance. The surface roughness also affects tooth feel. For example, polished teeth have a clean, smooth, and slick feel.

Numerous dentifrice compositions use precipitated silicas as abrasives. Precipitated silicas are noted and described in U.S. Pat. No. 4,340,583, Jul. 20, 1982, to Wason, EP Patent 535,943A1, Apr. 7, 1993, to McKeown et al., PCT Application WO 92/02454, Feb. 20, 1992 to McKeown et al., U.S. Pat. No. 5,603,920, Feb. 18, 1997, and U.S. Pat. No. 5,716,601, Feb. 10, 1998, both to Rice, and U.S. Pat. No. 6,740,311, May 25, 2004 to White et al.

While providing effective cleaning of teeth, precipitated silicas in oral care compositions may present compatibility problems with key formula actives, such as oxidizing agents like peroxide. Peroxides have been proven effective for oral cosmetic purposes, such as tooth whitening, as well as for the treatment of gingivitis, sensitivity, oral lesions, erosion, cavities, calculus, periodontitis, herpetic stomatitis, plaque, and for relieving bad breath. But often, due to compatibility problems, hydrogen peroxide and other oxidizing agents are not effectively delivered to the user. These compatibility problems have been shown to be directly related to surface properties of precipitated silicas such as surface area, number of hydroxyl groups, and porosity, and to the purity of the silica.

Furthermore, the rheology and subsequent stability of such rheology of oral care composition, particularly dentifrices, is very challenging to formulate. The composition must not be too thick so it can easily dispense out of a tube but thick enough to stand up on a toothbrush without sinking into the bristles. The viscosity of the oral composition must remain stable over time as not to continue to thicken so the oral composition remains easy to dispense during the shelf life. Once dispensed from a container, the oral composition should not be stringy or sticky as to be messy for a consumer to use. The oral composition must also easily disperse once in the mouth and create foam. It is also desired that the oral composition not stick to a sink or leave difficult to remove hard dried residue. In addition to balancing the viscosity and shear thinning to formulate acceptable rheology, the oral composition must also be stable and keep active ingredients, such as fluoride, available.

In addition to the above requirement for a consumer desired oral composition, it is also desired that oral composition be relatively easy to process. The oral composition must have the desired rheology and shelf stability as described above but also be viscous enough to quickly fill the oral composition into a container. It is also desired that the process not require special equipment and that the time to process not be long. It is also desired that the process be economical. Typically, oral compositions are thickened with polymeric thickeners. Polymeric thickeners may require a hydration step which can limit processing flexibility and cause aeration problems. It is also desired that the thickening system of an oral composition be low cost and comprise commonly available ingredients.

Based on the foregoing, there is an ongoing need for improved or care compositions with good rheology containing abrasives and hydrogen peroxide in a stable system for oral compositions. A need exists for an abrasive system that has good compatibility with oral care actives, such as oxidizing agents, while providing effective and safe cleaning and polishing of dental tissue. The compositions of the present invention may provide such benefits. The present invention therefore relates to oral compositions comprising sodium fluoride or MFP, hydrogen peroxide, fused silica or calcium pyrophosphate or dicalcium phosphate, and a gel network.

There is also the need for economical and convenient processes in making oral compositions. None of the existing art provides all of the advantages and benefits of the present invention.

### SUMMARY OF THE INVENTION

The present invention is directed to an oral composition containing a gel network, sodium fluoride or MFP, hydrogen peroxide, abrasive, and a protection system.

The present invention is further directed to an oral composition containing substantially aqueous oral care composition containing hydrogen peroxide and having improved stability, wherein said said composition comprises:
a) from about 0.1% to about 6%, by weight of the composition, of hydrogen peroxide;
b) a fluoride ion source selected from sodium fluoride and sodium monofluorophosphate;
c) from about 0.1% to about 40%, by weight of the composition, of an abrasive wherein the abrasive is selected from the group consisting of fused silica, calcium pyrophosphate, dicalcium phosphate, and mixtures thereof;
d) from about 0.05% to about 3%, by weight of the composition, of a protection system, wherein said protection system consists essentially of:
   i) from about 0.01% to about 1%, by weight of the composition, of sodium acid pyrophosphate;
   ii) from about 0.01% to about 1%, by weight the composition, of disodium phosphate; and
   iii) from about 0.01% to about 1%, by weight of the composition, of phosphoric acid;
      and
e) from about 5% to about 25%, by weight of the composition, of a gel network consisting of fatty alcohols and surfactant
   wherein the composition further comprises less than about 5% total, by weight of the composition, of water-miscible humectants selected from glycerin, sorbitol, diglycerin, and triglycerin; and
   wherein the composition has a pH of from about 3 to about 6.

The present invention is further directed to such compositions wherein said gel network system comprises:
i) from about 5% to about 25%, by weight of the composition, of cetyl alcohol;
ii) from about 5% to about 25%, by weight of the composition, of stearyl alcohol; and
iii) from about 0.3% to about 5%, by weight of the composition, of sodium lauryl sulfate.

The present invention is further directed to such compositions wherein the composition comprises from about 0.5% to about 6.0% by weight of the composition, of hydrogen peroxide.

The present invention is further directed to such compositions wherein the composition comprises from about 1% to about 40%, by weight of the composition, of the abrasive and the abrasive is selected from calcium pyrophosphate, dicalcium phosphate, and mixtures thereof.

The present invention is further directed to such compositions wherein the fused silica is selected from acid washed fused silica.

The present invention is further directed to such compositions wherein the fused silica has a median particle size of from about 3 microns to about 15 microns, wherein 90% of the particles have a particle size of about 50 microns or less.

The present invention is further directed to such compositions wherein the fluoride ion source is sodium monofluorophosphate.

The present invention is further directed to such compositions wherein the composition is substantially free of glycerin, sorbitol, diglycerin, and triglycerin.

The present invention is further directed to such compositions wherein the composition is substantially free of precipitated silica.

The present invention is further directed to such compositions wherein the composition comprises greater than about 35%, even 40%, by weight of the composition, of water.

The present invention is further directed to such compositions wherein the composition comprises from about 0.1% to about 2% by weight of the composition, of the protection system.

The present invention is further directed to such compositions wherein the composition comprises from about 5% to about 20%, by weight of the composition, of the gel network.

The present invention is further directed to such compositions wherein the composition further comprises an additional oral care ingredient selected from anti-calculus agents, anti-bacterial agents, anti-microbial agents, deposition polymers, food colorings, dyes, flavors, and mixtures thereof.

The present invention is further directed to such compositions wherein the composition further comprises about 0.1% to about 5%, by weight of the composition, of an additional surfactant, wherein the additional surfactant is selected from anionic, cationic, zwitterionic, amphoteric, and non-ionic surfactants, and mixtures thereof.

The present invention is further directed to such compositions wherein the additional surfactant is selected from anionic surfactants and mixtures thereof.

The present invention is further directed to an aqueous oral care composition containing hydrogen peroxide and having improved stability, wherein said composition consists essentially of:

a) from about 1% to about 3%, by weight of the composition, of hydrogen peroxide;

b) a fluoride ion source selected from sodium fluoride and sodium monofluorophosphate;

c) from about 2% to about 15%, by weight of the composition, of an abrasive selected from the group consisting of acid-washed fused silica, calcium pyrophosphate, dicalcium phosphate, and mixtures thereof;

d) from about 40% to about 80%, by weight of the composition, of water;

e) from about 0.1% to about 1%, by weight of the composition, of a protection system, wherein said protection system comprises:
i) from about 0.05% to about 0.5%, by weight of the composition, of sodium acid pyrophosphate;
ii) from about 0.05% to about 0.4%, by weight of the composition, of disodium phosphate; and
iii) from about 0.05% to about 0.5%, by weight of the composition, of phosphoric acid;

f) from about 7% to about 15%, by weight of the composition, of a gel network system, wherein said gel network system comprises:
i) from about 3.5% to about 7.5%, by weight of the composition, of cetyl alcohol;
ii) from about 3.5% to about 7.5%, by weight of the composition, of stearyl alcohol; and
iii) from about 0.7% to about 2%, by weight of the composition, of anionic surfactant;

g) from about 0.1% to about 3%, by weight of the composition, of an additional anionic surfactant;

wherein the composition further comprises less than about 5% total, by weight of the composition, of water-miscible humectants selected from glycerin, sorbitol, diglycerin, and triglycerin and precipitated silica; and
wherein the composition has a pH of from about 3 to about 6.

The present invention is further directed to such compositions wherein the surfactant of the gel network and the additional anionic surfactant are both sodium lauryl sulfate.

The present invention is further directed to such compositions wherein the composition is substantially free of additional abrasives.

The present invention is further directed to methods of using the compositions above for cleaning teeth wherein said methods comprise the step of applying the composition to the teeth with a tooth cleaning implement.

### BRIEF DESCRIPTION OF THE DRAWINGS

None

### DETAILED DESCRIPTION OF THE INVENTION

While the specification concludes with claims that particularly point out and distinctly claim the invention, it is believed the present invention will be better understood from the following description.

### DEFINITIONS

The term “comprising” as used herein means that steps and ingredients other than those specifically mentioned can be added. This term encompasses the terms “consisting of” and “consisting essentially of.” The compositions of the present invention can comprise, consist of, and consist essentially of the essential elements and limitations of the invention described herein, as well as any of the additional or optional ingredients, components, steps, or limitations described herein.

The term “effective amount” as used herein means an amount of a compound or composition sufficient to induce a positive benefit, an oral health benefit, and/or an amount low enough to avoid serious side effects, i.e., to provide a reasonable benefit to risk ratio, within the sound judgment of a skilled artisan.

The term “teeth” as used herein refers to natural teeth as well as artificial teeth or dental prosthesis.

The term “polymer” as used herein shall include materials whether made by polymerization of one type of monomer or made by two (i.e., copolymers) or more types of monomers.

The term “water soluble” as used herein means that the material is soluble in water in the present composition. In general, the material should be soluble at 25° C. at a concentration of 0.1% by weight of water, preferably at 1%, more preferably at 5%, more preferably at 15%.

The term “phase” as used herein means a mechanically separate, homogeneous part of a heterogeneous system.

The term “substantially non-hydrated” as used herein means that the material has a low number of surface hydroxyl groups or is substantially free of surface hydroxyl groups. It may also mean that the material contains less than about 5% total water (free or/and bound).

The term “majority” as used herein means the greater number or part; a number more than half the total.

The term “median” as used herein means the middle value in a distribution, above and below which lie an equal number of values.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

### Aqueous Oral Care Composition

The term “oral composition” as used herein means a product that in the ordinary course of usage is retained in the oral cavity for a time sufficient to contact some or all of the dental surfaces and/or oral tissues for purposes of oral activity. The oral composition of the present invention may be in various forms including toothpaste, dentifrice, tooth gel, tooth powders, tablets, rinse, subgingival gel, foam, mouse, chewing gum, lipstick, sponge, floss, prophy paste, petrolatum gel, or denture product. The oral composition may also be incorporated onto strips or films for direct application or attachment to oral surfaces, or incorporated into floss.

The term “dentifrice” as used herein means paste, gel, powder, tablets, or liquid formulations, unless otherwise specified, that are used to clean the surfaces of the oral cavity. In one embodiment, the compositions herein are toothpaste compositions.

The oral care compositions set forth herein are aqueous compositions. As used herein, “aqueous” means containing water. The amount of water may vary but typically is at least 20%, by weight of the composition.

In one embodiment, the composition contains at least about 30% water, alternatively at least 35%, alternatively at least 40% water, still alternatively at least 50% water or 60%, by weight of the composition, of water. In one embodiment, the compositions contain, from about 40% to about 80%, by weight of the composition, of water, alternatively from about 50% to about 75%, still alternatively from about 60% to about 75%, by weight of the composition, of water.

### Hydrogen Peroxide

The compositions according to the present invention contain from about 0.1% to about 6%, by weight of the composition, of hydrogen peroxide. In one embodiment, the composition contains from about 0.5% to about 3.0%, alternatively from about 1% to about 3%, by weight of the composition, of hydrogen peroxide.

The combination of hydrogen peroxide with fused silica is generally described in detail in U.S. Patent Application Publication No. 2010/0135931, published Jun. 3, 2010 and assigned to the Procter & Gamble Company, incorporated herein by reference.

### Fluoride

The compositions according to the present invention contain a fluoride ion source selected from sodium fluoride, sodium monofluorophosphate, and mixtures thereof. The fluoride ion is present in an amount sufficient to provide the monograph fluoride ion concentration in the composition at 25° C., and/or in one embodiment can be used at levels of from about 0.0025% to about 5.0% by weight of the composition, in another embodiment from about 0.005% to about 2.0% by weight of the composition, to provide anticaries effectiveness. In one embodiment, the composition consists essentially of a fluoride ion source selected from sodium fluoride. In another embodiment, the composition contains less than 1%, alternatively less than 0.5%, alternatively less than 0.001%, alternatively is substantially free of (i.e. contains no readily measurable level of) stannous fluoride.

### Abrasive

The compositions according to the present invention contain from about 0.1% to about 40% by weight of the composition, alternatively from about 1% to about 30%, alternatively from about 5% to about 25%, by weight of the composition, of abrasive. In one embodiment, the abrasive is selected from fused silica, calcium phosphates and mixtures thereof. In one embodiment, the abrasive is selected from fused silica, calcium pyrophosphate, dicalcium phosphate, and mixtures thereof. In another embodiment, the abrasive is selected from acid washed fused silica, calcium pyrophosphate, dicalcium phosphate, and mixtures thereof. In still another embodiment, the abrasive is selected from calcium pyrophosphate, dicalcium phosphate and mixtures thereof.

Fused Silica

The compositions according to the present invention may contain from about 0.1% to about 25%, by weight of the composition, of fused silica. In one embodiment, the composition contains from about 1% to about 15%, by weight of the composition, of fused silica. In another embodiment, the composition comprises less than about 2%, by weight of the composition, of fused silica in combination with another abrasive selected from calcium pyrophosphate, dicalcium phosphate, and mixtures thereof.

In one embodiment, the composition contains less than 1%, still alternatively no fused silica and includes an abrasive selected from calcium pyrophosphate, dicalcium phosphate, and mixtures thereof.

Fused silica materials useful in oral care compositions are set forth in more detail in US Patent Application Publication 2010/0135928, published Jun. 3, 2010 and assigned to the Procter & Gamble Company, herein incorporated by reference.

Fused silica is a high-purity amorphous silicon dioxide. It is sometimes referred to as fused quartz, vitreous silica, silica glass, or quartz glass. Fused silica is a type of glass, which, typical of glasses, lacks long-range order in its atomic structure. But the optical and thermal properties of fused silica are unique from those of other glasses, as fused silica typically has more strength, thermal stability, and ultraviolet transparency. For these reasons, fused silica is known to be used in situations such as semiconductor fabrication and laboratory equipment.

Without being bound by theory, it is believed that the fused silica, with its low BET specific surface area, low porosity, and low number of surface hydroxyl groups, is less reactive than precipitated silica. Consequently, the fused silica may adsorb less of other components, such as flavors, actives, or cations, leading to better availability for these other components. For example, dentifrices incorporating fused silica have superior stability and availability for fluoride, zinc, other cationic antibacterials, and hydrogen peroxide. Fused silica formulated in a dentifrice composition may result in at least about 50%, 60%, 70%, 80%, or 90% compatibility with cations or other components.

The shape of the particles of fused silica may be classified as either angular or spherical, or a combination of shapes, depending on the type of manufacturing process. Additionally, the fused silica may also be milled to reduce particle size. Spherical particles include any particle where the whole particle is mostly rounded or elliptical in shape. Angular particles include any particle that is not spherical, including polyhedral shapes. The angular particles may have some rounded edges, some or all sharp edges, some or all jagged edges, or a combination. The particle shape of the fused silica can impact its abrasivity.

Compositions that comprise spherical fused silica, that is, wherein at least 25% of the fused silica particles are spherical, have certain advantages. Due to the rounded edges, the spherical fused silica may be less abrasive. This means that the PCR to RDA ratio can be improved while still providing good cleaning. Also, spherical fused silica may be used at higher levels without being too abrasive. The spherical fused silica may also be used in combination with the angular fused silica, or silica wherein at least about 25% of the particles are angular. This could help lower costs, while still delivering good cleaning with acceptable abrasivity. In embodiments that have both angular and spherical fused silica, the amount of angular fused silica may be from about 1% to about 10%, by weight of the composition. In some embodiments wherein at least 25% of the fused silica particles are spherical, the RDA may be less than 150, in other embodiments less than 120. In some embodiments wherein at least 25% of the fused silica particles are spherical, the PCR to RDA ratio may be at least about 0.7, at least about 0.8, at least about 0.9, or at least about 1.0. In some of those embodiments, the median particle size of the fused silica is from about 3.0 microns to about 15.0 microns.

Examples of spherical fused silicas include Spheron P1500 and Spheron N-2000R, made by Japanese Glass Company, and Sun-Sil 130NP.

In some embodiments, the particle size of the fused silica may be optimized for cleaning In some embodiments, the median particle size of the fused silica may be from about 3 microns to about 15 microns, wherein 90% of the particles have a particle size of about 50 microns or less. In other embodiments, the median particle size may be from about 5 microns to about 10 microns, wherein 90% of the particles have a particle size of about 30 microns or less. In other embodiments, the median particle size may be from about 5 microns to 10 microns, wherein 90% of the particles have a particle size of about 15 microns or less.

In some embodiments, the particle size of the fused silica may be reduced to focus on polishing and anti-sensitivity benefits. In some embodiments, the fused silica may have a median particle size of from about 0.25 micron to about 5.0 microns, from about 2.0 microns to about 4.0 microns, or from about 1.0 micron to about 2.5 microns. In some embodiments, 10% of the fused silica particles may have a particle size of about 2.0 microns or less. In some embodiments, 90% of the fused silica particles may have a particle size of about 4.0 microns or less. In other embodiments, the particle size may be relatively large to be part of a prophy paste or some other non-daily use paste. In some embodiments, the fused silica may have a median particle size of at least about 7 microns and wherein the composition has a PCR of at least about 100. In other embodiments, the median particle size may be from about 7 microns to about 20 microns.

In some embodiments, an additional abrasive may be used, selected from the group consisting of pumice, perlite, precipitated silica, calcium carbonate, rice hull silica, silica gels, aluminas, other phosphates including orthophosphates, polymetaphosphates, pyrophosphates, other inorganic particulates, and mixtures thereof. In embodiments with the larger particle size, the fused silica may be from about 1% to about 10%, by weight of the composition. Some embodiments may be essentially free of surfactant, fluoride, or any oral care active. Some embodiments may have a flavoring agent. Some embodiments are methods of cleaning and polishing dental enamel by comprising an oral care composition wherein the median particle size is at least about 7 microns and the composition has a PCR of at least about 100.

One suitable type of fused silica is Teco-Sil 44CSS, which is available from C-E Minerals Products. Also available from C-E Minerals Products are fused silicas designated as Teco-Sil 44C, Teco-Sil T10, and TecoSpere A. Other suitable fused silicas include R61000, available from Jiangsu Kaida Silica and Spheron N-2000R and Spheron P1500, available from JGC, Japanese Glass Company. Others include RST 2500, RG 1500, and RG 5, available from Lianyungang Ristar Electronic Materials, SO-C5 and SO-C4, available from Adamatech, Fuserex AS-1, available from Tatsumori, FS 30 and FS-2DC, available from Denki Kagaku Kogyou, Min-Sil 325F, available from Minco, and Sunsil-130NP, available from Sunjin, and a fused silica from Shin-Etsu.

Acid Washed Fused Silica

In one embodiment, the fused silica is acid washed fused silica. In one embodiment, the general procedure for acid washing silica powder entails mixing the silica in a dilute acid solution, isolating the silica, using repeated water rinses, and finally drying the silica back to a crisp powder for ease of handling.

The specific steps that may be followed in this execution are:
1) Prepare a dilute acid solution using 1 part concentrated Hydrochloric Acid to 199 parts DI water, creating a solution ˜0.06M concentration.
2) Mix about 1 part silica to 4 parts dilute acid solution for about an hour.
3) Centrifuge for 10 minutes at 10,000 RPMs.
4) Decant and discard supernatant.
5) Fill centrifuge container with DI water, vortex to re-suspend silica, and repeat steps 3 and 4.
6) Repeat the Step 5 rinse procedure three more times.
7) Measure the pH of the supernatant of the final rinse to confirm that it measures between 6-7.
8) Dry the wet silica to a free flowing powder, as for example in a 60 C oven overnight.

Other acids may be used, such as phosphoric acid or mixtures of acids. Further, different concentrations of acids may also be used.

### Protection System

The compositions according to the present invention contain at least about 0.05%, by weight of the composition, of a protection system. In one embodiment, the composition contains from about 0.05% to about 2%, by weight of the composition, of the protection system. In one embodiment, the composition comprises from about 0.1% to about 1.5%, alternatively from about 0.1% to about 1%, by weight of the composition, of the protection system.

As used herein, “protection system” refers to a combination of materials that, without being limited by theory, when included in the compositions of the present invention will reduce degradation of hydrogen peroxide in the composition and improve overall product stability.

Examples of protection system materials useful herein include metal chelating and sequestering agents (such as, phosphates, pyrophosphates, and ethylenediaminetetraacetic acid and derivatives) chelating acids (such as, phosphoric acid, citric acid, lactic acid, malic acid, fumaric acid, tartaric acid), antioxidants (such as, mono- and dihydroxybenzenes and their analogs).

In one embodiment, the protection system may comprise, consist essentially of or consist of a mixture of two or more of sodium acid pyrophosphate, disodium phosphate and phosphoric acid. In another embodiment, the protection system may contain one or more of these three materials in combination with other materials.

Example protection systems useful herein may contain from 0.01% to about 1%, by weight of the composition, of sodium acid pyrophosphate; from 0.01% to about 1%, by weight of the composition, of disodium phosphate, and/or from 0.01% to about 1%, by weight of the composition, of phosphoric acid. In one embodiment, the protection system contains from about 0.1% to about 1%, alternatively from about 0.1% to about 0.5%, by weight of the composition, of sodium acid pyrophosphate; from about 0.05% to about 1%, alternatively from about 0.1% to about 0.4%, by weight the composition, of disodium phosphate; and/or from about 0.05% to about 1%, alternatively from about 0.075% to about 0.3%, by weight of the composition, of phosphoric acid.

### Gel Network System

The compositions according to the present invention contain from about 5% to about 25%, by weight of the composition, of a gel network system. In one embodiment, the composition comprises from about 5% to about 20%, alternatively from about 7% to about 15%, by weight of the composition, of the gel network.

In one embodiment, the composition is free of polymeric thickeners such as carrageenan.

Gel network systems and processes for making oral care compositions containing gel networks are disclosed in more detail in U.S. Patent Application Publication No. 2009/0246151 A1, published on Oct. 1, 2009 and assigned to the Procter & Gamble Company, as well as in U.S. Patent Application Publication No. US 2010-0135929 published Jun. 3, 2010 and assigned to the Procter & Gamble Company, all of which are incorporated herein by reference.

The gel network systems useful herein include from about 5% to about 25%, alternatively from about 3.5% to about 7.5%, by weight of the composition, of cetyl alcohol (fatty amphiphile); from about 5% to about 25%, alternatively from about 3.5% to about 7.5%, by weight of the composition, of stearyl alcohol (fatty amphiphile); and from about 0.3% to alternatively from about 0.7% to about 2%, about 5%, by weight of the composition, of sodium lauryl sulfate (swelling surfactant).

The presence of the gel network in the oral composition in the form of the ELD can be confirmed by means known to one skilled in the art, such as X-ray analysis, optical microscopy, electron microscopy, and differential scanning calorimetry. Methods of X-ray analysis and differential scanning calorimetry are described in the Examples below.

In an embodiment of the present invention, the weight ratio of the fatty amphiphile to the swelling surfactant in the gel network component is greater than about 1:5, preferably from about 1:3 to about 100:1, more preferably greater than about 1:1 to about 20:1, and even more preferably greater than about 2:1 to about 10:1.

Fatty Amphiphile

The gel network component of the present invention comprises at least one fatty amphiphile. As used herein, “fatty amphiphile” refers to a compound having a hydrophobic tail group of R₁ as defined below and a hydrophilic head group which does not make the compound water soluble (immiscible), wherein the compound also has a net neutral charge at the pH of the oral composition. The term “water soluble”, as used herein, means that the material is soluble in water in the present composition. In general, the material should be soluble at 25° C. at a concentration of 0.1% by weight of the water, preferably at 1%, more preferably at 5%, more preferably at 15%.

The fatty amphiphile of the present invention may be characterized as a compound having a Hydrophilic-Lipophilic Balance (“HLB”) of 6 or less. The HLB, as used herein, is the standard HLB according to Griffin, J. Soc. Cosm. Chem., vol. 5, 249 (1954). If using a mixture of fatty amphiphiles, it is desired that the mixture have a HLB of from about 1 to about 6 and preferably from about 1 to about 3. Therefore, fatty amphiphile having an HLB above 6 can be used if it is mixed with another fatty amphiphile having a lower HLB. In forming the fatty amphiphile dispersion, a low HLB is desired so that a lamellar phase does not form with the dispersing surfactant. The lower HLB means that the fatty amphiphiles will not be soluble in the water and reduced swelling of the fatty amphiphiles.

According to the present invention, suitable fatty amphiphiles, or suitable mixtures of two or more fatty amphiphiles, preferably have a melting point of at least about 40° C. In some embodiments, it is preferred that the melting point be at least about 50° C. or greater than about 55° C. or greater than about 60° C. The melting point, as used herein, may be measured by a standard melting point method as described in U.S. Pharmacopeia, USP-NF General Chapter <741>“Melting range or temperature”. The melting point of a mixture of two or more materials is determined by mixing the two or more materials at a temperature above the respective melt points and then allowing the mixture to cool. If the resulting composite is a homogeneous solid below about 45° C., then the mixture has a suitable melting point for use in the present invention. A mixture of two or more fatty amphiphiles, wherein the mixture comprises at least one fatty amphiphile having an individual melting point of less than about 45° C., still is suitable for use in the present invention provided that the composite melting point of the mixture is at least about 45° C.

According to the present invention, suitable fatty amphiphiles have a hydrophobic tail group of R₁. As used herein, R₁ is an alkyl, alkenyl (containing up to 3 double bonds), alkyl aromatic, or branched alkyl group of C₁₂-C₇₀ length. Non-limiting examples of alkyl, alkenyl, or branched alkyl groups suitable for the fatty amphiphiles of the present invention include lauryl, tridecyl, myristyl, pentadecyl, cetyl, heptadecyl, stearyl, arachidyl, behenyl, undecylenyl, palmitoleyl, oleyl, palmoleyl, linoleyl, linolenyl, arahchidonyl, elaidyl, elaeostearyl, erucyl, isolauryl, isotridecyl, isomyristal, isopentadecyl, petroselinyl, isocetyl, isoheptadecyl, isostearyl, isoarachidyl, isobehnyl, gadoleyl, brassidyl, and technical-grade mixture thereof.

As used herein, R₁ also may be a branched alkyl group prepared by alkaline condensation of alcohols to give higher molecular weight, branched isoalcohols. These branched isoalcohols are referred to in the art as Guerbet alcohols.

R₁ may be alkyl, alkenyl or branched carbon chains of vegetable origin, such as wheat germ, sunflower, grape seed, sesame, maize, apricot, castor, avocado, olive, soybean, sweet almond, palm, rapeseed, cotton seed, hazelnut, macadamia, karite, jojoba, alfalfa, poppy, pumpkinseed, sesame, cucumber, blackcurrant, evening primrose, millet, barley, quinoa, rye, safflower, candlenut, passion flower or musk rose oil, and karite butter.

Suitable fatty amphiphiles of the present invention also have a hydrophilic head group which does not make the compound water soluble, such as in compounds having an HLB of 6 or less. Non-limiting examples of classes of compounds having such a hydrophilic head group include fatty alcohols, alkoxylated fatty alcohols, fatty phenols, alkoxylated fatty phenols, fatty amides, alkoxylated fatty amides, fatty amines, fatty alkylamidoalkylamines, fatty alkoxyalted amines, fatty carbamates, fatty amine oxides, fatty acids, alkoxylated fatty acids, fatty diesters, fatty sorbitan esters, fatty sugar esters, methyl glucoside esters, fatty glycol esters, mono, di & tri glycerides, polyglycerine fatty esters, alkyl glyceryl ethers, propylene glycol fatty acid esters, cholesterol, ceramides, fatty silicone waxes, fatty glucose amides, and phospholipids.

To form the gel network component of the present invention, individual fatty amphiphile compounds or combinations of two or more different fatty amphiphile compounds may be selected. The following provides non-limiting examples of classes of compounds from which one or more fatty amphiphiles suitable for use in the present invention may be selected.

Fatty Alcohols/Alkoxylated Fatty Alcohol Ethers

Fatty amphiphiles of the present invention may be selected from fatty alcohol compounds or alkoxylated fatty alcohol ether compounds according to the following formula:

R₁—(OR₂)ₖ—OH

wherein R₁ is as described above; R₂ is a C₁-C₅ carbon chain which may be branched or hydroxy substituted; and k is a number ranging from about 0 to about 5.

The fatty alcohols useful herein are those having from about 12 to about 60 carbon atoms, preferably from about 16 to about 60 carbon atoms. These fatty alcohols may be straight or branched chain alcohols and may be saturated or unsaturated. Non-limiting examples of suitable fatty alcohols include cetyl alcohol, stearyl alcohol, arachidyl alcohol, behenyl alcohol, eicosyl alcohol, C20-40 alcohols, C30-50 alcohols, C40-60 alcohols, and mixtures thereof.

Suitable alkoxylated fatty alcohol ethers include addition products of 1 to 5 mol of ethylene oxide with a linear fatty alcohol having about 12 to about 60 carbon atoms, which are all adducts obtainable by the known industrial oxyethylation processes. Also suitable are the polyethylene oxide condensates of alkyl phenols, for example, the condensation products of alkyl phenols having an alkyl group containing from about 12 to about 60 carbon atoms in either a straight chain or branched chain configuration, with ethylene oxide, wherein the ethylene oxide is present in amounts equal to from about 1 to about 5 moles of ethylene oxide per mole of alkyl phenol. Further suitable alkoxylated fatty alcohol ethers include those derived from the condensation of ethylene oxide with the product resulting from the reaction of propylene oxide and ethylene diamine products.

Non-limiting examples of suitable alkoxylated fatty alcohol ethers include steareth-2, beheneth-2, beheneth-5, beheneth-10, C20-40 Pareth-3, C20-40 Pareth-10, C30-50 Pareth-3, and C30-50-Pareth-10.

In one embodiment, a combination of fatty alcohols such as cetyl and stearyl alcohol is preferred. The ratio of cetyl to stearyl alcohol can be from about 4:1 to about 1:4, preferably from about 2:1 to about 1:2, and in some embodiments 1:1.

Other Fatty Amphiphiles

Fatty amphiphiles of the present invention may be selected from di-fatty ethers, fatty amides including fatty alkanolamides and fatty alkoxylated amides, fatty carbamates, fatty alkylamido alkylamines, fatty amines including fatty alkanolamines and fatty alkoxylated amines, fatty amine oxides, fatty acids or alkoxylated fatty acids, fatty esters, fatty phosphorus compounds fatty sorbitan derivatives, sucrose polyesters, alkyl sulfoxides, and combinations thereof.

Suppressing Materials

It may be desired to add a suppressing material to the fatty amphiphile dispersion. The function of the suppressing agent is to suppress the swelling of the fatty amphiphile while it is in the dispersion and before it is mixed with the swelling surfactant. The suppressing material will help keep the viscosity low and help to prevent lamellar phase formulation. Suppressing materials include any material that helps control swelling of the fatty amphiphile. Specific materials include soluble and insoluble polar materials with dialetric constants of from about 6 to about 20 and typically from about 6 to about 13. Suitable materials include polar oils such as flavor oils, and coolants, salts, and combinations thereof. The suppressing material is optionally added to the fatty amphiphile dispersion. The suppressing material can be added in an amount of from about 0% to about 10%, commonly from about 0.1% to about 8%, and from about 1% to about 5% by weight of the final oral composition.

Swelling Surfactant

The gel network component of the present invention also comprises a swelling surfactant. As used herein, “swelling surfactant” refers to one or more surfactants which are combined with the fatty amphiphile dispersion to form the gel network of the present invention. The swelling surfactant is not intended to be present in the pre-made fatty amphiphile dispersion and it may be desired to have the fatty amphiphile dispersion be essentially free of swelling surfactants. The swelling surfactant is typically water soluble. The swelling surfactant may be characterized as a compound having a Hydrophilic-Lipophilic Balance (“HLB”) of 6 or more and typically from about 8 to about 30. The HLB, as used herein, is the standard HLB according to Griffin, J. Soc. Cosm. Chem., vol. 5, 249 (1954). Preferably, the surfactant will be reasonably stable and foam throughout a wide pH angle.

The oral compositions of the present invention comprise swelling surfactant as part of gel network phase in an amount from about 0.01% to about 15%, preferably from about 0.1% to about 10%, and more preferably from about 0.3% to about 5%, by weight of the oral composition. In some embodiments, a diluted solution of surfactant in water is utilized. In one embodiment, the amount of surfactant is chosen based on the level of foaming desired in the oral composition and on the irritation caused by the surfactant. Once the level of surfactant is chosen, then the level of fatty amphiphile that forms a gel network is chosen.

Suitable swelling surfactants include anionic, zwitterionic, amphoteric, cationic, and nonionic surfactants. In one embodiment, anionic surfactants are preferred. The swelling surfactants may be a combination of more than one type of surfactants, such as an anionic, nonionic, and zwitterionic surfactant.

Anionic surfactants useful herein include the water-soluble, water-miscible salts of alkyl sulfate having from 8 to 20 carbon atoms in the alkyl radical (e.g., sodium alkyl sulfate) and the water-soluble or water-miscible salts of sulfonated monoglycerides of fatty acids having from 8 to 20 carbon atoms. Preferred anionic surfactants for use as swelling surfactants of the present invention include sodium lauryl sulfate, sodium lauryl sarcosinate, sodium cocoyl methyl taurate, sodium monoglyceride sulfate, sodium cetaryl sulfate, potassium cocoyl glycinate, sodium lauryl phosphate, sodium lauryl lactylate, sodium lauryl sulfoacetate, sodium lauryl glutamate, sodium lauryl isethionate, sodium laureth carboxylate, sodium dodecyl benzenesulfonate, and combinations thereof. In one embodiment, sodium lauryl sulfate is a preferred swelling surfactant. Many suitable anionic surfactants are disclosed by Agricola et al., U.S. Pat. No. 3,959,458, issued May 25, 1976.

Nonionic surfactants useful herein can be broadly defined as compounds produced by the condensation of alkylene oxide groups (hydrophilic in nature) with an organic hydrophobic compound which may be aliphatic or alkyl-aromatic in nature. Nonlimiting examples of suitable nonionic surfactants include polyoxyethylene sorbitan esters (sold under the trade name Tweens), polyoxyl 40 hydrogenated castor oil, fatty alcohol ethoxylates, ethylene oxide condensates of aliphatic alcohols, long chain tertiary amine oxides, long chain tertiary phosphaine oxides, lauryl glucoside (sold under the trade name Plantaren 1200 UP) and long chain dialkyl sulfoxides. Suitable nonionic surfactants with a HLB of 7 or more include sucrose laurate, sucrose cocoate, sucrose stearate; Steareth 20, 21, or 100, and PEG 20 Sorbitan Monostearate (commercially available as Tween 60).

Amphoteric surfactants suitable as a swelling surfactant in the present invention can be broadly described as derivatives of aliphatic secondary and tertiary amines in which the aliphatic radical can be a straight chain or branched and wherein one of the aliphatic substituents contains from about 8 to about 18 carbon atoms and one contains an anionic water-solubilizing group, such as carboxylate, sulfonate, sulfate, phosphate, or phosphonate. Other suitable amphoteric surfactants are betaines, such as cocamidopropyl betaine, lauryl dimethyl betaine (sold under the trade name Macat LB), cetyl dimethyl betaine, and cocoamphodiacetate. Additional amphoteric surfactants and nonionic surfactants can be found in Gieske et al., U.S. Pat. No. 4,051,234, issued Sep. 27, 1977. Examples of suitable cationic surfactants include cetyl pyridinium chloride, coamidopropyl PG dimonium chloride phosphate (Phospholipid CDM), myristylamidopropyl PG dimonium chloride phosphate (Phospholipid PTM), stearamidopropyl PG dimonium chloride phosphate (Phospholipid SV), steapyrium chloride (Catemol WPC), and other suitable cationic materials.

More than one surfactant of the above specified types may be used for the swelling surfactant of the present invention.

Another swelling surfactant or surfactant of any type may also be added to the oral carrier phase of the oral composition. This surfactant may not be part of the gel network as it does not participate in forming the gel network structure. The surfactant in the oral carrier phase may provide enhanced foaming or a different foaming profile. The surfactant added to the oral carrier phase may also aid in modifying viscosity and changing the flavor display.

Gel networks, generally, are further described by G. M. Eccleston, “Functions of Mixed Emulsifiers and Emulsifying Waxes in Dermatological Lotions and Creams”, Colloids and Surfaces A: Physiochem. and Eng. Aspects 123-124 (1997) 169-182; and by G. M Eccleston, “The Microstructure of Semisolid Creams”, Pharmacy International, Vol. 7, 63-70 (1986).

### Additional Surfactant

The compositions according to the present invention may contain from about 0.1% to about 5%, by weight of the composition, of additional surfactant. In one embodiment, the compositions contains from about 0.1% to about 3%, by weight of the composition, of additional surfactant.

Surfactants, also commonly referred to as sudsing agents, may aid in the cleaning or foaming of the dentifrice composition. The additional surfactant may be the same surfactant that is used to form the gel network. Without being limited by theory, once sufficient surfactant is present in the composition to form the gel network, additional surfactant may be added and may provide cleaning benefits. Suitable surfactants are those which are reasonably stable and foam throughout a wide pH range. Additional surfactants useful herein include anionic, cationic, zwitterionic, amphoteric, and non-ionic surfactants, and mixtures thereof.

In one embodiment, the additional surfactant is selected from anionic surfactants and mixtures thereof. In one embodiment, the additional surfactant is sodium lauryl sulfate.

Examples of anionic surfactants useful herein include the water-soluble or water-miscible salts of alkyl sulfates having from 8 to 20 carbon atoms in the alkyl radical (e.g., sodium alkyl sulfate) and the water-soluble or water-miscible salts of sulfonated monoglycerides of fatty acids having from 8 to 20 carbon atoms. Sodium lauryl sulfate (SLS) and sodium coconut monoglyceride sulfonates are examples of anionic surfactants of this type. Examples of other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458, issued May 25, 1976. In some embodiments, the oral care composition may comprise an anionic surfactant at a level of from about 0.025% to about 9%, from about 0.05% to about 5% in some embodiments, and from about 0.1% to about 1% in other embodiments.

Another suitable surfactant is one selected from the group consisting of sarcosinate surfactants, isethionate surfactants and taurate surfactants. Preferred for use herein are alkali metal or ammonium salts of these surfactants, such as the sodium and potassium salts of the following: lauroyl sarcosinate, myristoyl sarcosinate, palmitoyl sarcosinate, stearoyl sarcosinate and oleoyl sarcosinate. The sarcosinate surfactant may be present in the compositions of the present invention from about 0.1% to about 2.5%, or from about 0.5% to about 2% by weight of the total composition.

Cationic surfactants useful in the present invention include derivatives of aliphatic quaternary ammonium compounds having one long alkyl chain containing from about 8 to 18 carbon atoms such as lauryl trimethylammonium chloride; cetyl pyridinium chloride; cetyl trimethylammonium bromide; di-isobutylphenoxyethyl-dimethylbenzylammonium chloride; coconut alkyltrimethylammonium nitrite; cetyl pyridinium fluoride; etc. Preferred compounds are the quaternary ammonium fluorides described in U.S. Pat. No. 3,535,421, Oct. 20, 1970, to Briner et al., where said quaternary ammonium fluorides have detergent properties. Certain cationic surfactants can also act as germicides in the compositions disclosed herein.

Nonionic surfactants that can be used in the compositions of the present invention include compounds produced by the condensation of alkylene oxide groups (hydrophilic in nature) with an organic hydrophobic compound which may be aliphatic or alkylaromatic in nature. Examples of suitable nonionic surfactants include the Pluronics, polyethylene oxide condensates of alkyl phenols, products derived from the condensation of ethylene oxide with the reaction product of propylene oxide and ethylene diamine, ethylene oxide condensates of aliphatic alcohols, acids, and esters, long chain tertiary amine oxides, long chain tertiary phosphine oxides, long chain dialkyl sulfoxides and mixtures of such materials.

Zwitterionic synthetic surfactants useful in the present invention include derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate.

Suitable betaine surfactants are disclosed in U.S. Pat. No. 5,180,577 to Polefka et al., issued Jan. 19, 1993. Typical alkyl dimethyl betaines include decyl betaine or 2-(N-decyl-N,N-dimethylammonio)acetate, coco betaine or 2-(N-coc-N,N-dimethyl ammonio)acetate, myristyl betaine, palmityl betaine, lauryl betaine, cetyl betaine, cetyl betaine, stearyl betaine, etc. The amidobetaines are exemplified by cocoamidoethyl betaine, cocoamidopropyl betaine, lauramidopropyl betaine and the like. The betaines of choice are preferably the cocoamidopropyl betaine and, more preferably, the lauramidopropyl betaine.

### Selected Water-Miscible Humectants & Precipitated Silica

In one embodiment, the compositions according to the present invention contain less than about 5% total, by weight of the composition, of the total amount of the water-miscible humectants selected from glycerin, sorbitol, diglycerin, and triglycerin, and precipitated silica. In one embodiment, the compositions contain less than about 3% total, alternatively less than about 1% total, by weight of the composition of the combination of water-miscible humectants selected from glycerin, sorbitol, diglycerin, and triglycerin and precipitated silica.

Without being limited by theory, it is believed that due to the manufacturing process for certain humectants and/or precipitated silica, that small amounts of metal impurities may be present and that such metal impurities may reduce the stability of the compositions set forth herein. It may be possible to purify such materials or use small amounts to avoid instability. However, in one embodiment, the compositions according to the present invention are substantially free of, that is, contain no readily measurable quantity, of the total amount of water-miscible humectants selected from glycerin, sorbitol, diglycerin, and triglycerin and precipitated silica.

In one embodiment, the compositions herein contain less than about 2%, alternatively less than about 1%, alternatively less than about 0.5%, alternatively less than about 0.1%, by weight of the composition, of glycerin, sorbitol, diglycerin, and/or triglycerin.

Precipitated Silica

Numerous dentifrice compositions use precipitated silicas as abrasives. Precipitated silicas are noted and described in U.S. Pat. No. 4,340,583, Jul. 20, 1982, to Wason, EP Patent 535,943A1, Apr. 7, 1993, to McKeown et al., PCT Application WO 92/02454, Feb. 20, 1992 to McKeown et al., U.S. Pat. No. 5,603,920, Feb. 18, 1997, and U.S. Pat. No. 5,716,601, Feb. 10, 1998, both to Rice, and U.S. Pat. No. 6,740,311, May 25, 2004 to White et al.

In one embodiment, the compositions herein contain less than about 2%, alternatively less than about 1%, alternatively less than about 0.5%, alternatively less than about 0.1%, by weight of the composition, of precipitated silica. In one embodiment, the compositions herein contain less than about 1%, alternatively less than about 0.5%, alternatively less than about 0.001%, by weight of the composition, of precipitated silica.

In one embodiment, the compositions herein contain less than about 1%, alternatively less than about 0.1%, by weight of the composition of other abrasives, meaning abrasives other than the fused silica abrasives set forth above.

### PH

The compositions according to the present invention have a pH of from about 3 to about 6. In one embodiment, the pH of the composition is from about 4 to about 6, alternatively from about 4.5 to about 5.5.

In one embodiment, pH modifiers, such as citric acid, may be used in the compositions herein to adjust the pH to a desirable level. Therefore, in one embodiment, the compositions herein contain citric acid. In one embodiment, the citric acid is included at a level suitable to reach the desired pH.

### Additional Oral Care Ingredient

In one embodiment, the composition contains from about 0.05% to about 20%, alternatively from about 0.5% to about 15%, alternatively from about 5% to about 10%, by weight of the composition, of an additional oral care ingredient selected from anti-calculus agents, anti-bacterial agents, anti-microbial agents, deposition polymers, food colorings, dyes, flavors, and mixtures thereof.

### Methods of Use

The compositions of the present invention are used in a conventional manner for cleansing the teeth. Generally, a method of using a dentifrice to cleanse the teeth comprises applying the composition of the present invention to a cleaning implement, such as a toothbrush, brushing the teeth for a period of time, and then rinsing the dentifrice from the mouth. From about 0.01 to about 3 grams of toothpaste is typically used.

### EXAMPLES

The oral care compositions illustrated in the following Examples illustrate specific embodiments of the oral compositions of the present invention, but are not intended to be limiting thereof. Other modifications can be undertaken by the skilled artisan without departing from the spirit and scope of this invention.

### Example I

### Toothpaste Compositions

Examples 1A to 1D, are shown in Table 1, below and are drawn to toothpaste compositions according to the present invention. The compositions may be made according to standard making procedures for gel network compositions known to one skilled in the art. Generally, to make the compositions, the gel network is formed first by combining the LANETTE W with the sodium lauryl sulfate powder and water and heated above melt temperature (above approximately 70° C.). As the mixture is then allowed to cool, the remaining ingredients are added. Hydrogen peroxide is added last at a temperature below approximately 30° C. The mixture is then allowed to cool to approximately ambient temperature.

TABLE 1

| | 1A | 1B | 1C | 1D |
|---|---|---|---|---|
| Ingredient | wt. % | wt. % | wt. % | wt. % |
| LANETTE W* | 10.75 | 15.0 | 18.0 | 7.0 |
| Sodium Lauryl | 0.55 | 1.9 | 1.25 | 1.25 |
| Sulfate Powder* | | | | |
| Sodium | 0.76 | | | |
| Monofluorophosphate | | | | |
| Sodium Fluoride | | 0.243 | 0.243 | 0.243 |
| Sucralose | 0.32 | 0.2 | 0.25 | 0.18 |
| Peppermint B Flavor | 0.5 | 1.0 | 1.25 | 1.75 |
| Phosphoric Acid | 0.05 | 0.10 | 0.18 | 0.02 |
| Disodium phosphate | 0.1 | 0.20 | 0.35 | 0.05 |
| Sodium acid | 0.05 | 0.30 | 0.5 | |
| pyrophosphate | | | | |
| TECOSIL-44CSS | | 10.0 | 1.0 | |
| (acid washed)* | | | | |
| TECOSIL-44CSS* | 10.0 | | | 5.0 |
| H2O2 (35% solution | 3.5 | 8.5 | 8.5 | 2.0 |
| in water) | | | | |
| Citric Acid | 0.38 | 0.38 | 0.44 | 0.28 |
| Water | Q.S. | Q.S | Q.S. | Q.S. |

LANETTE W is a commercially available mixture of approximately 45% cetyl alcohol, 45% stearyl alcohol and 10% sodium lauryl sulfate, available from COGNIS, Manheim, Germany.
Sodium Lauryl Sulfate powder is commercially available from Stepan, Chicago, Ill., USA.
TECOSIL-44CSS is a fused silica powder commercially available from CE Minerals, Tennessee, USA. The material may be used as provided by the supplier or may be acid washed before using by the methods set forth herein above.

### Example II

### Toothpaste Compositions

Examples 2E to 2J, are shown in Table 2, below and are drawn to toothpaste compositions according to the present invention. The compositions may be made according to standard making procedures for gel network compositions known to one skilled in the art. Generally, to make the compositions, the gel network is formed first by combining the LANETTE W with the sodium lauryl sulfate powder and water and heated above melt temperature (above approximately 70° C.). As the mixture is then allowed to cool, the remaining ingredients are added. Hydrogen peroxide is added last at a temperature below approximately 30° C. The mixture is then allowed to cool to approximately ambient temperature.

TABLE 2

| | 2E | 2F | 2G | 2H | 2I | 2J |
|---|---|---|---|---|---|---|
| Ingredient | wt. % | wt. % | wt. % | wt. % | wt. % | wt. % |
| LANETTE W | 16.0 | 16.0 | 16.0 | 16.0 | 16.0 | 16.0 |
| NaF | | | | | 0.243 | |
| Sodium | 1.14 | 1.14 | 1.14 | 0.76 | | 1.14 |
| monofluorophosphate | | | | | | |
| Calcium | 15.0 | 15.0 | 20.0 | | 15.0 | 10.0 |
| Pyrophosphate | | | | | | |
| Fused silica (acid | | 1.0 | | | | |
| washed) | | | | | | |
| Dicalcium phosphate | | | | 15.0 | | 5.0 |
| anhydrous | | | | | | |
| Sucralose | 0.25 | 0.25 | 0.25 | 0.25 | 0.25 | 0.25 |
| Flavor | 1.5 | 1.5 | 1.5 | 1.5 | 1.5 | 1.5 |
| Sodium acid | 0.3 | 0.3 | 0.3 | 0.3 | 0.3 | 0.3 |
| pyrophosphate | | | | | | |
| Disodium phosphate | 0.2 | 0.2 | 0.2 | 0.2 | 0.2 | 0.2 |
| H2O2 (35% soln) | 8.57 | 8.57 | 8.57 | 8.57 | 8.57 | 8.57 |
| Phosphoric Acid (est | 0.237 | 0.2 | 0.3 | 0.2 | 0.18 | 0.3 |
| 0.1-0.4%) | | | | | | |
| Water | Q.S. | Q.S. | Q.S. | Q.S. | Q.S. | Q.S. |

LANETTE W is a commercially available mixture of approximately 45% cetyl alcohol, 45% stearyl alcohol and 10% sodium lauryl sulfate, available from COGNIS, Manheim, Germany.
The calcium pyrophosphate is commercially available from Prayon, Belgium.
The fused silica may be TECOSIL-44CSS, a fused silica powder commercially available from CE Minerals, Tennessee, USA. The material is acid washed before using by the methods set forth herein above.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aqueous oral care composition containing hydrogen peroxide and having improved stability, wherein said composition comprises:
a) at least 40%, by weight of the composition, of water;
b) from about 0.1% to about 6%, by weight of the composition, of hydrogen peroxide;
c) a fluoride ion source selected from sodium fluoride and sodium monofluorophosphate;
d) from about 0.1% to about 40%, by weight of the composition, of an abrasive wherein the abrasive is selected from the group consisting of fused silica, calcium pyrophosphate, dicalcium phosphate, and mixtures thereof;
e) from about 0.05% to about 3%, by weight of the composition, of a protection system,
wherein said protection system consists essentially of:
i) from about 0.01% to about 1%, by weight of the composition, of sodium acid pyrophosphate;
ii) from about 0.01% to about 1%, by weight the composition, of disodium phosphate; and
iii) from about 0.01% to about 1%, by weight of the composition, of phosphoric acid;
and
f) from about 5% to about 25%, by weight of the composition, of a gel network consisting of fatty alcohols and surfactant
wherein the composition further comprises less than about 5% total, by weight of the composition, of water-miscible humectants selected from glycerin, sorbitol, diglycerin, and triglycerin; and
wherein the composition has a pH of from about 3 to about 6.

2. An aqueous oral care composition according to claim 1, wherein said gel network system comprises:
i) from about 5% to about 25%, by weight of the composition, of cetyl alcohol;
ii) from about 5% to about 25%, by weight of the composition, of stearyl alcohol; and
iii) from about 0.3% to about 5%, by weight of the composition, of sodium lauryl sulfate.

3. An aqueous oral care composition according to claim 1, wherein the composition comprises from about 0.5% to about 3.0%, by weight of the composition, of hydrogen peroxide.

4. An aqueous oral care composition according to claim 1, wherein the composition comprises from about 1% to about 40%, by weight of the composition, of the abrasive and the abrasive is selected from calcium pyrophosphate, dicalcium phosphate, and mixtures thereof.

5. An aqueous oral care composition according to claim 1, wherein the fused silica is selected from acid washed fused silica.

6. An aqueous oral care composition according to claim 1, wherein the composition comprises less than 1%, by weight of the composition, of fused silica.

7. An aqueous oral care composition according to claim 1, wherein the fluoride ion source is sodium monofluorophosphate.

8. An aqueous oral care composition according to claim 1, wherein the composition is substantially free of glycerin, sorbitol, diglycerin, and triglycerin.

9. An aqueous oral care composition according to claim 1, wherein the composition is substantially free of precipitated silica.

10. An aqueous oral care composition according to claim 1, wherein the composition comprises greater than about 40%, by weight of the composition, of water.

11. An aqueous oral care composition according to claim 1, wherein the composition comprises from about 0.1% to about 1.5%, by weight of the composition, of the protection system.

12. An aqueous oral care composition according to claim 1, wherein the composition comprises less than 0.01% of polymeric thickeners.

13. An aqueous oral care composition according to claim 1, wherein the composition comprises from about 5% to about 20%, by weight of the composition, of the gel network.

14. An aqueous oral care composition according to claim 1, wherein the composition further comprises an additional oral care ingredient selected from anti-calculus agents, anti-bacterial agents, anti-microbial agents, deposition polymers, food colorings, dyes, flavors, and mixtures thereof.

15. An aqueous oral care composition according to claim 1, wherein the composition further comprises about 0.1% to about 5%, by weight of the composition, of an additional surfactant, wherein the additional surfactant is selected from anionic, cationic, zwitterionic, amphoteric, and non-ionic surfactants, and mixtures thereof.

16. An aqueous oral care composition according to claim 15, wherein the additional surfactant is selected from anionic surfactants and mixtures thereof.

17. An aqueous oral care composition containing hydrogen peroxide and having improved stability, wherein said composition consists essentially of:
a) from about 1% to about 3%, by weight of the composition, of hydrogen peroxide;
b) a fluoride ion source selected from sodium fluoride and sodium monofluorophosphate;
c) from about 2% to about 15%, by weight of the composition, of an abrasive selected from the group consisting of acid-washed fused silica, calcium pyrophosphate, dicalcium phosphate, and mixtures thereof;
d) from about 40% to about 80%, by weight of the composition, of water;
e) from about 0.1% to about 1%, by weight of the composition, of a protection system, wherein said protection system comprises:
i) from about 0.05% to about 0.5%, by weight of the composition, of sodium acid pyrophosphate;
ii) from about 0.05% to about 0.4%, by weight of the composition, of disodium phosphate; and
iii) from about 0.05% to about 0.5%, by weight of the composition, of phosphoric acid;
f) from about 7% to about 15%, by weight of the composition, of a gel network system, wherein said gel network system comprises:
i) from about 3.5% to about 7.5%, by weight of the composition, of cetyl alcohol;
ii) from about 3.5% to about 7.5%, by weight of the composition, of stearyl alcohol; and
iii) from about 0.7% to about 2%, by weight of the composition, of anionic surfactant;
g) from about 0.1% to about 3%, by weight of the composition, of an additional anionic surfactant;
wherein the composition further comprises less than about 5% total, by weight of the composition, of water-miscible humectants selected from glycerin, sorbitol, diglycerin, and triglycerin and precipitated silica; and
wherein the composition has a pH of from about 3 to about 6.

18. An aqueous oral care composition according to claim 17, wherein the surfactant of the gel network and the additional anionic surfactant are both sodium lauryl sulfate.

19. An aqueous oral care composition according to claim 17 wherein the composition is free of polymeric thickeners.

20. A method of using the composition according to claim 1 for cleaning teeth wherein said method comprises the step of applying the composition to the teeth with a tooth cleaning implement.
